(19) United States
(12) Patent Application Publication
(10) Pub. No.: US 2005/0182269 A1
(43) Pub. Date: Aug. 18, 2005

(54) Process for the preparation of 3-cyano-1-naphthoic acid and some analogues thereof

(76) Inventors: Ian Ashworth, Stalybridge (GB); Martin Bowden, Huddersfield (GB); Bruce Dembofsky, Wilmington, DE (US); Daniel Levin, Toronto (CA)

Correspondence Address: WHITE & CASE LLP;PATENT DEPARTMENT, 1155 AVENUE OF THE AMERICAS, NEW YORK, NY, 10036, US

(21) Appl. No.: 10/518,300

(22) PCT Filed: Jun. 18, 2003
(86) PCT No.: PCT/SE03/01045

(30) Foreign Application Priority Data
Jun. 20, 2002 (SE) 0201938-8

(51) Int. Cl. C07C 253/30

(52) U.S. Cl. 558/410

(57) ABSTRACT

The present invention is related to a process for the preparation of 3-cyano-1-naphthoic acid and some analogues thereof of formula (1), the intermediate 1-halo-3-cyano naphthalene and some analogues thereof used in this process and a process for the preparation of said intermediate.

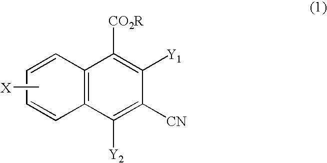

### FIELD OF THE INVENTION

The present invention is related to a process for the preparation of 3-cyano-1-naphthoic acid and some analogues thereof, the intermediate 1-halo-3-cyano naphthalene and some analogues thereof used in this process and a process for the preparation of said intermediate.

### BACKGROUND OF THE INVENTION

The compound 3-cyano-1-naphthoic acid is previously described in Jeffrey S. Albert et al; “Design, Synthesis, and SAR of Tachykinin Antagonist Activity”, J. Med. Chem, Vol. 45, no. 18, 2002, p. 3972-3983, p. 3973 Scheme 2; p. 3980-3981, no 18-20, Richtzenhain Hermann et al: “Substitution reactions with metalloorganic compounds. IV. The Grignardization of methoxyl-containing aromatic nitrites”; STN International, File CASREACT, Accession no. 44:10012, & Chem. Ber (1949), 82; 408-17, WO 01/77069, WO 00/59873, WO 00/20003, WO 00/20389, WO 02/12168, WO 01/77089 and WO 00/02859 and a process for the preparation of the same is previously described in Jeffrey S. Albert et al; “Design, Synthesis, and SAR of Tachykinin Antagonist Activity”, J. Med. Chem, Vol. 45, no. 18, 2002, p. 3972-3983, p. 3973 Scheme 2; p. 3980-3981, no 18-20, WO 01/77069, WO 00/59873, WO 00/20003, WO 00/20389, WO 02/12168 and WO 01/77089. This process is unattractive for commercial manufacture on account of toxic process effluent arising from use of mercury salt to achieve regioselective decarboxylation, low through process yield and operationally unattractive bromination in concentrated nitric acid.

### BRIEF DESCRIPTION OF THE INVENTION

The present invention refers to a process for preparing the compound of formula (1) 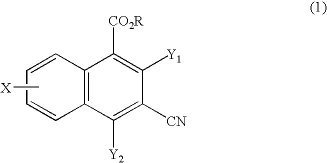
wherein X and/or Y₁ and/or Y₂ are independently H, cyano, nitro, trifluoromethoxy, trifluoromethyl, alkoxy, or alkyl and R is H or alkyl either
a) in the case where R═H, by metallo-dehalogenation followed by carboxylation of a compound of formula (12) 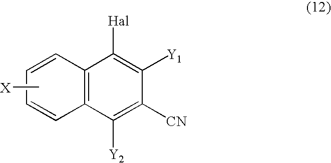
   wherein X, Y₁ and Y₂ are as defined above, and Hal is Br, I or Cl or
b) in the case where R═H or alkyl, by palladium mediated carbonylation of a compound of formula (12) 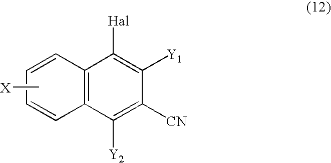
   with the proviso that the compound 1-iodo-3-cyano-2-methoxynaphthalene is excluded, followed by solvolysis.

Furthermore the present invention refers to a compound of formula (12) 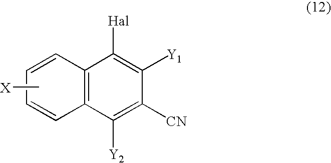
wherein X and/or Y₁ and/or Y₂ are independently H, cyano, nitro, trifluoromethoxy, trifluoromethyl, alkoxy, or alkyl and Hal is Br, I or Cl, which is a compound not previously described and which is a key intermediate in the preparation of the compound of formula (1, R═X═Y₁═Y₂═H) and moreover to a process for preparing a compound of formula (12, Y₁═Y₂═X═H).

Moreover the present invention refers to some other intermediates that may be used in the process for preparing the compound of formula (1, R═X═Y₁═Y₂═H), namely the compound of formula (20) 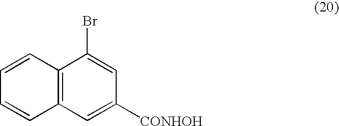
the compound of formula (18) 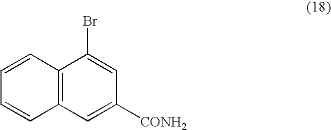
3-bromo-5-coumalonitrile (27) 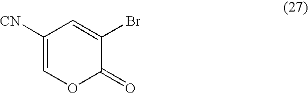
and the compound of formula (63)

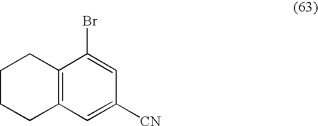

### DETAILED DESCRIPTION OF THE INVENTION

The process for preparing the compound of formula (1) 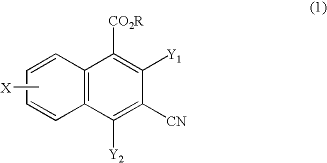
wherein X and/or Y₁ and/or Y₂ are independently H, cyano, nitro, trifluoromethoxy, trifluoromethyl, alkoxy, or alkyl and R is H or alkyl is carried out either
a) (in the case where R═H) by metallo-dehalogenation followed by carboxylation of a compound of formula (12) 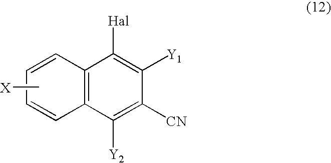
   wherein X, Y₁ and Y₂ are as defined above, and Hal is Br, I or Cl or
b) (in the case where R═H or alkyl) by palladium mediated carbonylation of a compound of formula (12) 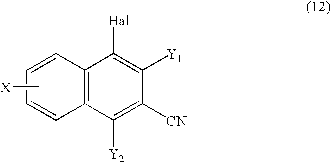
   with the proviso that the compound 1-iodo-3-cyano-2-methoxynaphthalene is excluded, followed by solvolysis.
   Metallo-Dehalogenation and Carboxylation of Compound of Formula (12)

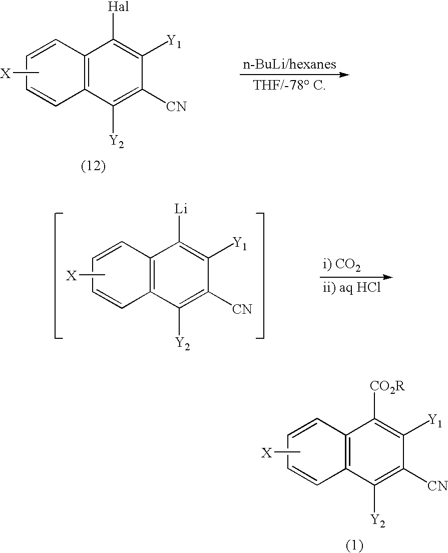

Metallo-dehalogenation and carboxylation may be carried out by treatment of compound (12) with alkyl-lithium reagent, e.g. “BuLi, in THF alone or in admixture with solvents like hexane at a temperature below −10° C., and preferably between −30° C. and −75° C., followed by reaction of the lithiated intermediate with CO₂ and subsequent acidification with e.g. HCl.
Palladium Mediated Carbonylation

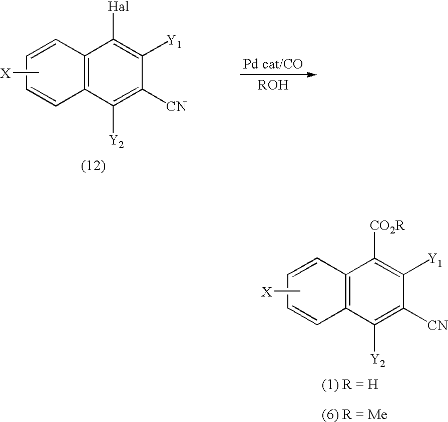

The halo-cyano-naphthalene (12) may be reacted with carbon monoxide under elevated pressure, for example between 5 bar and 100 bar, in a solvent such as methanol with an organic base such as triethylamine catalysed by palladium with or without additional phosphine ligand such as triphenyl phosphine or bis-diphenylphosphino propane. The active palladium catalyst can be generated in situ from palladium salts such as palladium (II) chloride or palladium bis(triphenylphosphine)palladium(In) chloride. The product (1) may be isolated by first of all removing solid residues by filtration and then extracting into aqueous and back into organic with pH control, followed by crystallisation from toluene. The product (6) may be isolated by removing solid residues by filtration followed by crystallisation from solvent.
The Process for Preparing the Compound of Formula (12. Y₁═Y₂═X═H) 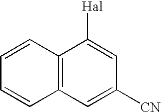
wherein Hal is Br, I or Cl
may be carried out by any of the following routes:
(i)
by
(a) treating malic acid (7) with oleum or alternative strongly acid dehydrating media to give coumalic acid (8);
(b) esterifying coumalic acid (8) to give a pyrone ester (9);
(c) brominating the pyrone ester (9) to give a 3-bromo coumalic ester (10);
(d) reacting the 3-bromo coumalic ester (10) with in situ generated benzyne followed by decarboxylation to give a bromonaphthoate (11); and
(e) converting/transforming the bromonaphthoate (11) to 1-bromo-3-cyano naphthalene (12, Y₁═Y₂═X═H)
   or (ii)
   by
(a) treating malic acid (7) with oleum or alternative strongly acid dehydrating media to give coumalic acid (8);
(b) converting coumalic acid (8) into coumalonitrile (25) and subsequently brominating to give 3-bromo-5-coumalonitrile (27); and then
(c) converting 3-bromo-5-coumalonitrile (27) into 1-bromo-3-cyano naphthalene (12, Y₁═Y₂═X═H)
   by cycloaddition of in situ generated benzyne, followed by subsequent decarboxylation
   or (iii)
   by
1a) cyanation of 1,2,3,4-tetrahydronaphthalene followed by bromination to give the compound of formula (63) 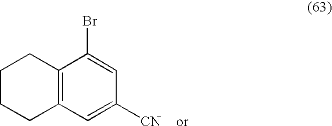
1b) bromination of 1,2,3,4-tetrahydronaphthalene followed by cyanodebromination, followed by bromination to give the compound of formula (63); or
1c) bromination of 1,2,3,4-tetrahydronaphthalene followed by metallation and carboxylation followed by conversion to the 6-cyano-1,2,3,4-tetrahydronaphthalene followed by bromination to give the compound of formula (63);
   followed by
2) oxidative aromatization of the compound of formula (63) into 1-bromo-3-cyano naphthalene (12, Y₁═Y₂═X═H);
   which are illustrated in the reaction schemes below.

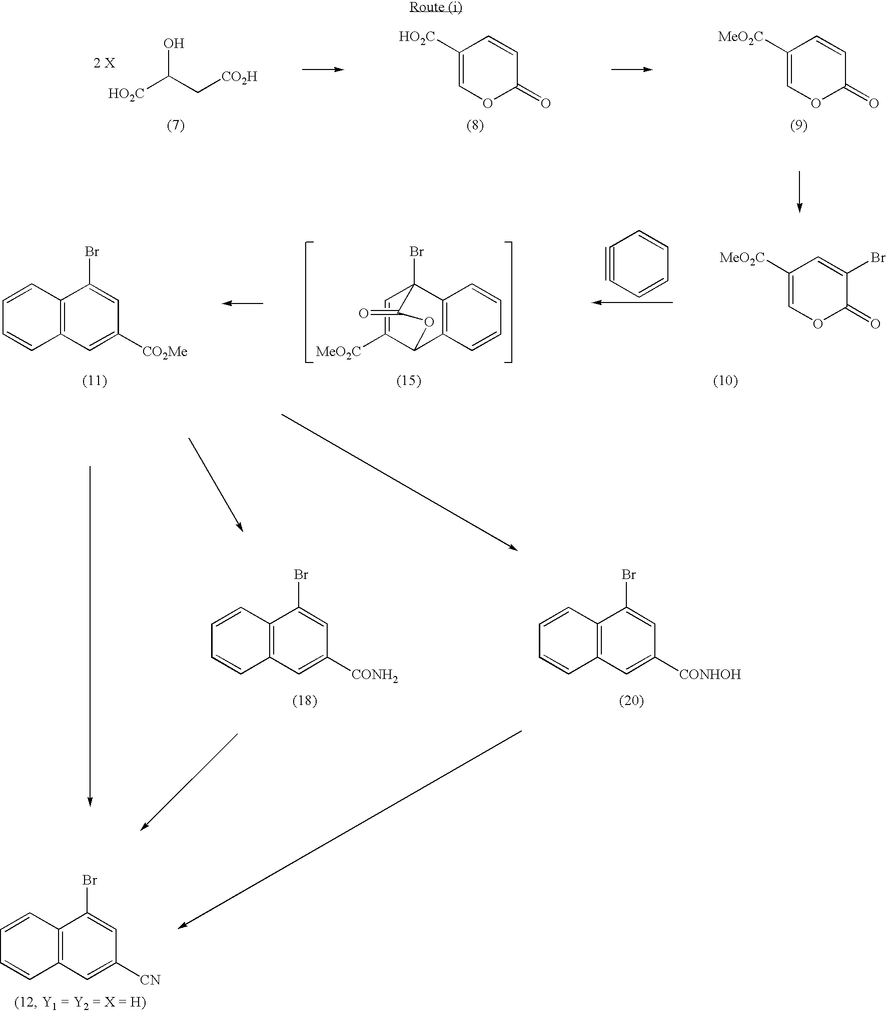

### Stage (a)—Coumalic Acid

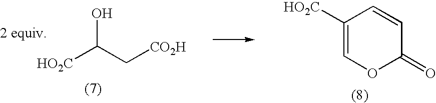

Oleum or alternative strongly acid dehydrating media is added to a suspension of malic acid in a strong acid e.g. H₂SO₄ at about 50° C. to 90° C., preferably at 75° C. to 85° C. Then the mixture is cooled and the product coumalic acid is filtered off.

Coumalic acid (8) is also commercially available.

### Stage (b)—Pyrone Ester

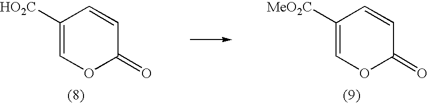

Diisopropylethylamine or other non-nucleophilic base (e.g. DBU) is added to a suspension of coumalic acid in NMP, dimethylsulphate (or else MeBr or MeI) and a non-nucleophilic base, e.g. DBU or ⁱPr₂Net, are added, and the reaction stirred at between 20° C. and 30° C. The reaction mass is diluted, e.g. with toluene, and drowned out into water followed by washing of the organic phase with aqueous bicarbonate and finally water. The solvent is removed by evaporation in vacuo and the crude product pyrone ester is purified by filtration isolation from the residual mother liquors.

### Stage (c)—3-Bromo Coumalic Acid

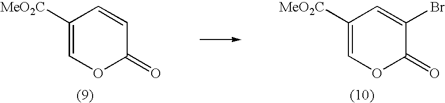

Pyrone ester is brominated, e.g. with pyridinium bromide perbromide (pyridinium tribromide) or Br₂ in glacial acetic acid to give 3-bromo coumalic acid.

### Stage (d)—Bromonaphthoate

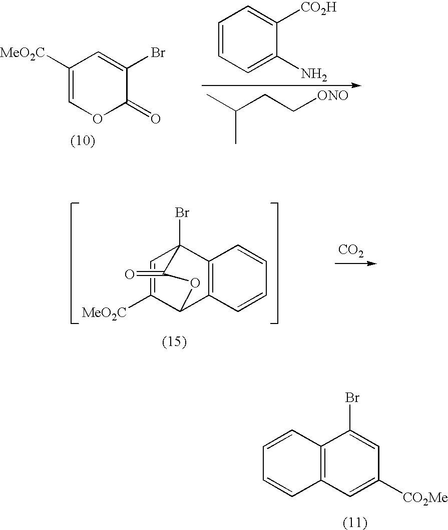

Isoamyl nitrite and a solution of anthranilic acid in e.g. ethylene glycol dimethyl ether are is added to a refluxing solution of a 3-bromo coumalic ester in e.g. ethylene glycol dimethyl ether in the presence of an acid, e.g. catalytic trichloroacetic acid. Benzene-2-diazonium carboxylate is formed by anthranilic acid diazotisation followed by in situ decomposition to give benzyne. The reactive benzyne undergoes [4+2] cycloaddition with the 3-bromo coumalic ester to give an intermediate (15), which then extrudes carbon dioxide to give the desired bromonaphthoate. Heating under reflux is continued, the reaction mass is then cooled to about 50° C., a solvent, e.g. toluene is added and the mixture then cooled to ambient. The solution is washed with dilute sodium hydroxide solution, sodium bisulphite solution, water, hydrochloric acid and water again. The solution is then concentrated in vacuo to give the crude bromonaphthoate product.

### Stage (e)—1-Bromo-3-Cyano naphthalene

Three methods for this transformation are possible:

### Method 1: Conversion to the Amide (18) Followed by Dehydration

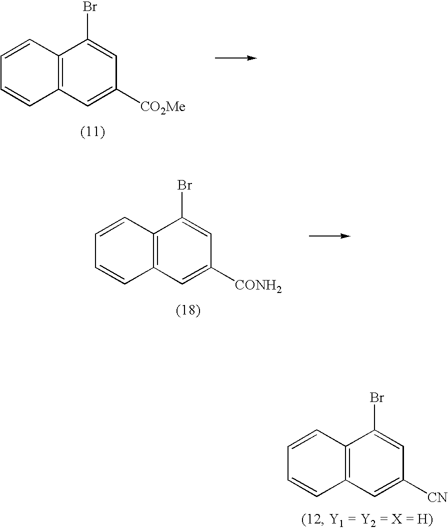

Bromonaphthoate (11) is heated with ammonia in the presence of a solvent, e.g. toluene; and a catalyst, e.g. KI, at a high temperature to give bromoamide (18) This is followed by dehydration by heating the bromoamide in a large excess of a dehydrating agent, e.g. SOCl₂, to give the compound of formula (12).

### Method 2: Conversion to the Hydroxamic Acid (20)

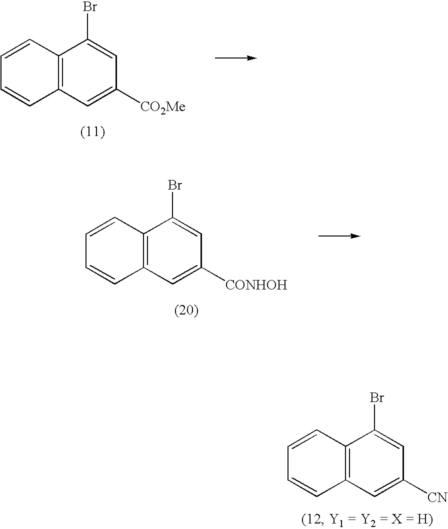

Preparation of hydroxamic acid (20) is achieved by reaction of a bromonaphthoate (11) with hydroxylamine, or a salt thereof, e.g. hydrochloride plus added base. Conversion of the hydroxamic acid (20) to 1-bromo-3-cyano naphthalene (12) is effected by dehydration, e.g. by treatment with PBr₃.

### Method 3: Direct Conversion of Bromonaphthoate (11) to 1-bromo-3-cyano naphthalene (12, X═Y₁═Y₂═H) with Me₂AlNH₂ (21)

The reagent for this transformation, dimethylaluminium amide, is prepared under strictly anhydrous conditions in an inert atmosphere by condensing anhydrous NH₃ into a solution of AlMe₃ at low temperature.

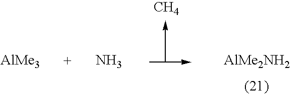

A solution of Me₂AlNH₂ solution is added to a solution of a bromonaphthoate in a high-boiling solvent, e.g. m-xylene, and the mixture is heated to reflux. Rapid conversion to the 1-bromo-3-cyano naphthalene (12) occurs and the product is isolated. 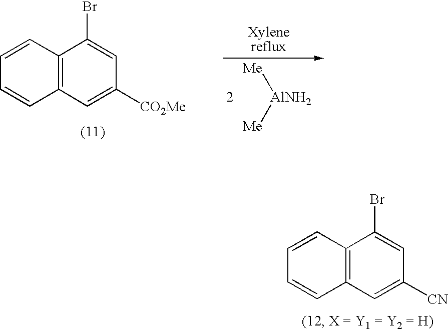
Route (ii)

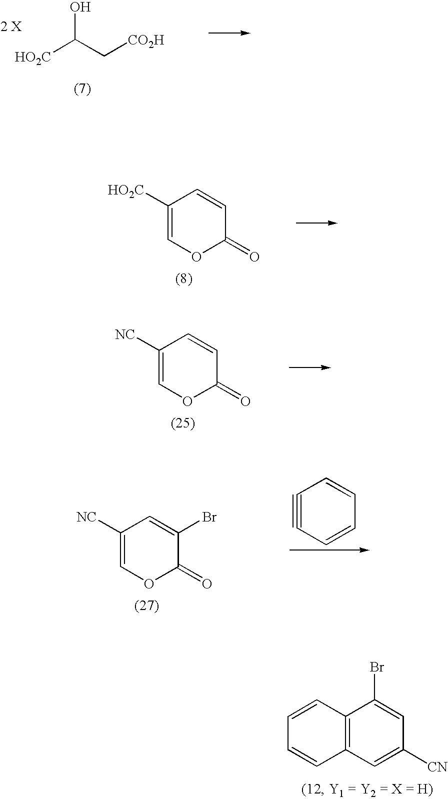

### Stage (a)—Coumalic Acid: See Route (i) Stage (a) Above

### Stage (b)—3-bromo-5-coumalonitrile

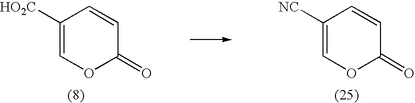

Coumalic acid (8) is converted to the corresponding nitrile (25) by conversion to the acid chloride (28) by reaction with a chlorinating agent, e.g. thionyl chloride, followed by reaction with sulfamide (H₂NSO₂NH₂).

Coumalonitrile (25) is brominated using a brominating agent, e.g. pyridinium bromide perbromide (PBPB) in a high-boiling solvent to give bromocoumalonitrile (27). The product is isolated from unreacted starting material by crystallisation.

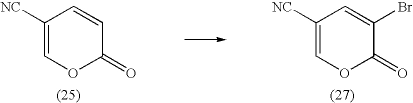

### Stage (c)—1-Bromo-3-Cyano naphthalene

Compound (27) is converted into compound (12) by cycloaddition of in situ generated benzyne, followed by subsequent decarboxylation e.g. by heating.

The presence of a cyano—rather than an ester group at the 5-position of pyrone ring does not affect the progress of the cycloaddition. 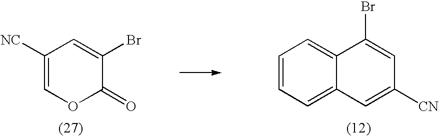
Route (iii)

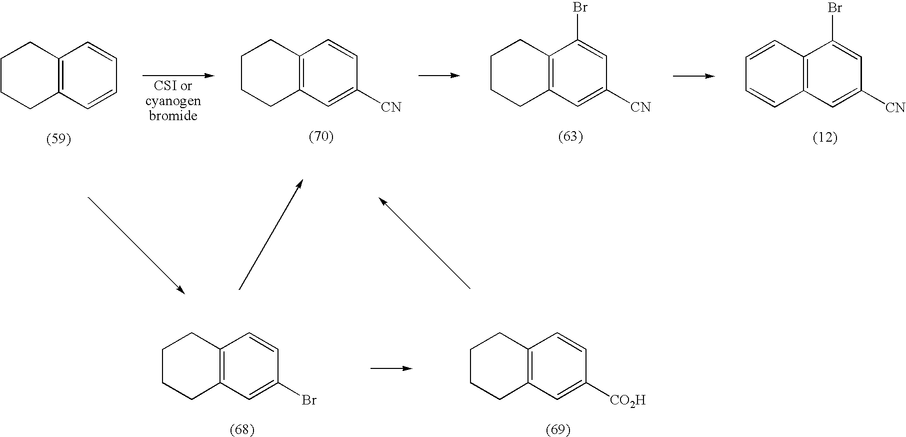

1,2,3,4-tetrahydronaphthalene (also known as Tetralin®) is cyanated to give cyanotetrahydronaphthalene (70), either directly by reaction with cyanogen bromide with aluminium chloride as catalyst in carbon disulphide, or via bromotetrahydronaphthalene (68), the resulting cyano tetrahydronaphthalene (70) is brominated to give bromocyanotetrahydronaphthalene (63) which is converted to bromocyanonaphthalene (12) by oxidative aromatisation.

Thus, tetrahydronaphthalene (59) is reacted with bromine, with added iodine as catalyst, the 6-bromo-1,2,3,4-tetrahydronaphthalene (plus regioisomers) is either a) cyanated by reaction with copper (I) cyanide in NMP at 130° C. for 48 h to give 6-cyano-1,2,3,4-tetrahydronaphthalene (70) or b) is lithiated by reaction with n-butyl lithium in THF at −78° C. followed by reaction with carbon dioxide and then dilute hydrochloric acid to furnish 5,6,7,8-tetrahydronaphthalene-2-carboxylic acid (69) along with its regioisomer from which tetrahydronaphthalene acid (69) is purified by repeated recrystallisation. This acid is converted to cyanonaphthalene (70) by conversion to acid chloride by reaction with thionyl chloride with a small amount of NMP as catalyst, followed by conversion to amide by reaction with ammonia, followed by amide dehydration, for example with PBr₃. 5,6,7,8-Tetrahydronaphthalene-2-carbonitrile (70) is brominated by reaction with bromine with catalytic ferric bromide in carbon tetrachloride to give bromonitrile (63).
Aromatisation of Substituted Tetralins

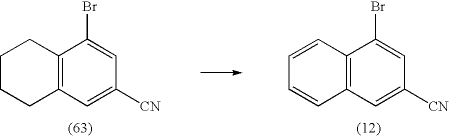

The aromatisation of the compound of formula (63) into the compound of formula (12) is carried out by heating the compound of formula (63) at a high temperature in the presence of a metal catalyst, e.g. Pd/C. Alternatively, the aromatisation may be carried out for example by stirring with elemental sulphur in a solvent at ambient temperature.

### PREPARATIONS

### Preparation 1

Conversion of malic acid to coumalic acid Oleum (287 g) is added dropwise over 2 h to a suspension of malic acid (200 g) in concentrated H₂SO₄ (313 g) at 75° C. and the resulting solution stirred for a further 4 h, maintaining temperature at 75° C. throughout. The mixture is cooled and then drowned out into ice-cold water over 1 h. After stirring for 15 min and standing overnight, the mixture is cooled to below 10° C. and the product is isolated by filtration to give coumalic acid (71 g, 95% purity, 65% yield) after washing and drying.

### Preparation 2

Conversion of coumalic acid to coumalic acid, methyl ester Diisopropylethylamine is added to a suspension of coumalic acid (115.5 g) in N-methylpyrrolidone (600 mL) at 25° C., dimethylsulphate (100.9 g) is added over 1 h and the reaction stirred at 25° C. for 2 h. The reaction mass is diluted with toluene, and extracted with water then bicarbonate and finally water. The toluene is removed in vacuo and the crude product pyrone ester is purified either by short path distillation or by crystallisation and trituration to give (after removal of residual solvent by evaporation in vacuo) the coumalic acid methyl ester (78.8 g, 99% purity, 64% yield).

### Preparation 3

Conversion of coumalic acid, methyl ester to 3-bromocoumalic acid, methyl ester A solution of pyrone ester (39 g, 95% purity) in acetic acid is added over 3.5 hr to a refluxing solution of pyridinium tribromide (105 g) in glacial acetic acid (233 g). The mixture is held at reflux (85° C.->107° C.) for 3 hr then cooled to ambient. Water is added and the crude product is isolated by filtration then washed with water. The crude product is purified by recrystallisation from toluene and iso-hexane to give 3-bromocoumalic acid, methyl ester (46 g, 82% yield).

### Preparation 4

Conversion of 3-bromocoumalic acid, methyl ester to methyl 4-bromo-2-naphthoate Isoamyl nitrite (24.2 g) and a solution of anthranilic acid (28.0 g) in ethylene glycol dimethyl ether (90 g) are added over 3 h to a refluxing solution of 3-bromo coumalic acid, methyl ester (23.3 g) in ethylene glycol dimethyl ether (135.8 g) in the presence of catalytic trichloroacetic acid (0.165 g). The reaction is refluxed for a further 1 hr after the end of addition to ensure complete reaction. The reaction mass is cooled to 50° C., toluene (279 g) is added and the mixture then cooled to ambient. The toluene solution is washed with sodium hydroxide solution (75 mL, 2M), sodium bisulphite solution (75 mL, 5%), water (75 mL), hydrochloric acid and water again. The toluene solution is then concentrated in vacuo to give methyl 4-bromo-2-naphthoate (30 g, 85% purity, 93% yield).

### Preparation 5

Conversion of methyl 4-bromo-2-naphthoate to 4-bromo-2-naphthonitrile Dimethylaluminium amide is prepared by the reaction of a solution of trimethylaluminium in toluene (150 mL, 2M) with excess anhydrous ammonia (25.5 g) at −78° C. Excess ammonia is removed by evaporation at 110° C. and the dimethylaluminium amide solution is then charged to a solution of the bromonaphthoate (39.8 g) in m-xylene (321.7 g) at 110° C. over 1 hour. The reaction is held at 110° C. for a further hour and then rapidly cooled to room temperature in ice. The reaction mass is drowned out into aqueous HCl (750, mL, 2M) over 1.5 hours at 5-10° C. The m-xylene solution is concentrated in vacuo to give the crude product, which is recrystallised from toluene/iso-hexane to give 4-bromo-2-naphthonitrile (18.9 g, 54% yield).

### Preparation 6

Conversion of methyl 4-bromo-2-naphthoate to 4-bromo-2-naphthonitrile via 4-bromo-2-naphthamide To a Carius tube equipped with small magnetic flea and protective outer metal casing is charged methyl 4-bromo-2-naphthoate (1.18 g), aqueous ammonia (9 ml), potassium iodide (0.075 g) and methanol (2 ml). The apparatus is assembled, and lowered into an oil bath at 130° C. The pressure rises to 4.25 bar. The mixture is heated with stirring under these conditions for 66 h, after which time the assembly is removed from the oil bath and allowed to cool to ambient temperature/pressure. The mixture is cooled to 0° C. to complete crystallisation, and filtered to remove the product. The product is dissolved in EtOAc (50 ml) and washed with 10% w/v aqueous Na₂CO₃ (2×10 ml). The organic layer is separated, dried MgO₄) and the solvent removed in vacuo to give the product 4-bromo-2-naphthamide as colourless prisms (0.38 g, 94% str by GC area, 33% yield,).

To a 10 ml 1-necked round-bottomed flask equipped with magnetic stirrer, condenser and inert atmosphere is charged 4-bromo-2-naphthamide (0.093 g) and thionyl chloride (2 ml). The mixture is heated under reflux for 18 h, and the excess thionyl chloride is removed in vacuo to afford the crude product 4-bromo-2-naphthonitrile as a yellow solid.

¹H nmr (CDCl₃): 8.15 (s, 1H, ArH), 8.24 (d, 1H, J=7.4 Hz, ArH), 7.90-7.62 (m, 4H, ArH).

MS: 233 (M⁺), 231 (M⁺), 152, 125, 76.

### Preparation 7

### Conversion of methyl 4-bromo-2-naphthoate to 4-bromo-2-naphthonitrile Via 4-bromo-N-hydroxy-2-naphthamide

To a 100 ml 2-necked round-bottomed flask equipped with magnetic stirrer, graduated pressure equalised dropping funnel and inert atmosphere is charged bromonaphthoate (2.69 g), hydroxylamine hydrochloride (2.78 g) and methanol (16 ml). 5 M Methanolic KOH (10 ml.) is added dropwise over 40 min to the vigorously stirred suspension at room temperature. An exotherm and an orange colouration is noted on each addition. The reaction mixture (beige suspension) is stirred at room temperature for 17 h after addition of base. The reaction mixture is concentrated to ca. half volume in vacuo (water bath <45° C.) and a 1:1 mixture of water/glacial acetic acid (50 ml) added with vigorous stirring. Stirring is continued for 40 min. and a further portion of 1.1 water/glacial acetic acid 20 ml) added when the suspension becomes too thick to stir. Stirring is continued for 1 h, and the product filtered off under reduced pressure and washed with cold water (3×15 ml). The product hydroxamic acid is dried in the vacuum oven at 70° C. to give 4-bromo-N-hydroxy-2-naphthamide as a beige powder (2.2 g, 76% str. by LC area, 76% yield,).

To an oven dried 250 ml 2-necked round-bottomed flask equipped with magnetic stirrer, condenser, septum and inert atmosphere is charged 4-bromo-N-hydroxy-2-naphthamide (2.0 g) and fluorobenzene (80 ml). Phosphorous tribromide (1.8 ml) is added dropwise over 10 min to the stirred suspension at room temperature and the mixture heated to reflux (85° C.) whereupon a clear orange solution is obtained. Reflux is continued for 18 h, and the solution allowed to cool. The crude reaction mixture is poured into saturated aqueous NaHCO₃ solution (50 ml) and the product extracted with toluene (3×50 ml). The combined organic extracts are washed with brine (50 ml) and the solvent removed in vacuo. The residue is crystallised from methanol to give the product 4-bromo-2-naphthonitrile as pale yellow prisms (0.73 g).

The ¹H nmr and mass spectra of the above end product corresponds to those previously 30 obtained.

### Preparation 8

### Conversion of Coumalic Acid to 3-bromo-2-oxo-2H-pyran-5-carbonitrile (3-bromocoumalonitrile) Via 2-oxo-2H-pyran-5-carbonitrile (coumalonitrile)

Coumalic acid (3.91 g) and thionyl chloride (31 ml) are charged to a 100 ml 2-neck round bottomed flask equipped with condenser, magnetic stirrer and inert atmosphere, and the suspension heated to reflux for 1 h. The clear yellow solution is allowed to cool, and the excess thionyl chloride removed in vacuo. Sulfamide (3.22 g) is added, and the solid mixture heated to 120° C. (bath temp.) for 1 h. The acid chloride melts after a few seconds, and HCl is vigorously evolved. After ca. 15 min, a red foam is obtained, which on further heating collapses to a dark red viscous oil. After 1 h, the reaction mixture has solidified. The reaction mixture is allowed to cool, and transferred to a separating funnel with 10% w/v aqueous NaHCO₃ solution (150 ml) (heating with the latter being necessary to remove the crude product from the flask). The product is extracted with CH₂Cl₂ (2×50 ml) and the combined organic layers washed with sat. NaCl solution (100 ml). The extracts are dried (MgSO₄) and the solvent removed in vacuo. The residue is purified by crystallisation from MeOH (2 ml) at 0° C. The product coumalonitrile is obtained as dark orange prisms (1.7 g). Coumalonitrile (2.0 g), pyridinum bromide perbromide (5.28 g), dimethoxy ethane (13 g) and toluene (12,98) are charged to a 100 ml 2-neck round bottomed flask equipped with condenser, magnetic stirrer and inert atmosphere, and heated under reflux for 4 h. The reaction mixture is poured into water (100 ml) and extracted with CH₂Cl₂ (3×100 ml). The extracts are dried (MgSO₄) and the solvent removed in vacuo. The residue is swirled with ether (20 ml) and the extracts decanted off. The residue is purified by crystallisation from acetone to give the 3-bromo-2-oxo-2H-pyran-5-carbonitrile as an orange powder (1.25 g, 81% str. by LC area, 31% yield).

¹H nmr (CDCl₃): 7.74 (d, 1H, J=2.5 Hz, Hₐ), 8.04 (d, 1H, J=2.2 Hz, H_{b}).

MS: 201 (M⁺), 199 (M⁺), 173, 171, 144, 142, 120, 64, 29.

### Preparation 9

Conversion of 3-bromo-2-oxo-2H-pyran-5-carbonitrile (3-bromocoumalonitrile) to 4-bromo-2-naphthonitrile Solutions of anthranilic acid (1.8 g, 12.8 mmol) in DME (10 ml) and isoamyl nitrite (1.54 g, 12.8 mmol) in DME (10 ml, 8.7 g) are added dropwise over 20 min to a stirred solution of 3-bromocoumalonitrile (1.15 g, 4.6 mmol) and trichloroacetic acid (0.047 g, 0.29 mmol) in DME (40 ml) held at reflux. The mixture is refluxed for a further 10 min, allowed to cool and poured into water (100 ml). The product is extracted with CH₂Cl₂ (2×50 ml) and the volatiles removed in vacuo. The product crystallises from the residual amyl alcohol at −20° C. and the dirty orange solid is collected by filtration in vacuo, and dried in the oven at 40° C. to give 4-bromo-2-naphthonitrile (0.81 g, 49% yield).

### Preparation 10

### Conversion of 1,2,3,4-Tetrahydronaphthalene to 5,6,7,8-tetrahydronaphthalene-2-carbonitrile

1,2,3,4-Tetrahydronaphthalene (3.3 g), aluminium chloride (6.7 g), cyanogen bromide (5.5 g) and carbon disulphide (70 ml) were heated together under reflux for 8 hours however this achieved negligible reaction, the mixture was accordingly concentrated by distilling out solvent at atmospheric pressure until the temperature of the reaction mixture rose to 60° C. Stirring was continued at 60° C. for 8 hours, the mixture was cooled, chloroform (100 ml) was added and the resulting mixture then added slowly to a stirred mixture of concentrated hydrochloric acid (3 g) and 50:50 ice water (150 ml) at 0° C. The resulting phases were separated, the aqueous layer was extracted with chloroform (2×100 ml), the combined organic phases were washed with saturated aqueous sodium bicarbonate solution (150 ml) and water (2×50 ml), they were dried (MgSO₄) and solvent removed by evaporation in vacuo to give crude product (3.8 g) comprising a 3:1 mixture of 5,6,7,8-tetrahydronaphthalene-2-carbonitrile and 5,6,7,8-tetrahydronaphthalene-1-carbonitrile. This was purified by distillation under reduced pressure to give 5,6,7,8-tetrahydronaphthalene-2-carbonitrile in 40% overall yield.

### Conversion of 5,6,7,8-tetrahydronaphthalene-2-carbonitrile to 4-bromo-5,6,7,8-tetrahydronaphthalene-2-carbonitrile

Bromine (2.5 g, 15.6 mmol) was added cautiously to a stirred mixture of 5,6,7,8-tetrahydronaphthalene-2-carbonitrile (2 g, 12 mmol) and ferric bromide (4.7 g, 15.6 mmol) in carbon tetrachloride (20 ml) at 10° C. The mixture was stirred at ambient temperature for 8 hours, it was worked up by adding to dilute aqueous hydrochloric acid and extracting with chloroform followed by removal of solvent by evaporation in vacuo to give the crude product as a brown oil (5.74 g, 45% purity by gc area, 86% yield). The product was purified by chromatography on silica gel using 1:9 ethyl acetate:hexane eluent to give 4-bromo-5,6,7,8-tetrahydronaphthalene-2-carbonitrile as a mixture of isomers.

### Preparation 11

### Conversion of 1,2,3,4-tetrahydronaphthalene to 5-bromo-1,2,3,4-tetrahydronaphthalene and 6-bromo-1,2,3,4-tetrahydronaphthalene

Bromine (66.1 g, 0.41 mol)) was added over 3 hours, with stirring at 5° C. to 10° C., to the 1,2,3,4-tetrahydronaphthalene (50 g, 0.374 mol) along with a small piece of iodine (0.25 g, 0.98 mmol). Stirring was continued at ambient temperature for 6 hours and the mixture was then poured slowly into a stirred saturated aqueous solution of sodium sulphite (200 ml) at 10° C. Stirring was continued for 15 minutes, the resulting mixture was extracted with methylene chloride (3×50 ml), the combined organic extracts were washed with water (200 ml), dried (MgSO₄) and solvent removed by evaporation in vacuo to give 5-bromo-1,2,3,4-tetrahydronaphthalene along with the 6-bromo-1,2,3,4-tetrahydronaphthalenec isomer (86 g, 89.7% purity of combined mono-brominated isomers present in circa 3:2 ratio, combined mono-bromo isomer yield 96%).

### Conversion of 5-bromo-1,2,3,4-tetrahydronaphthalene and 6-bromo-1,2,3,4-tetrahydronaphthalene to 5,6,7,8-tetrahydronaphthalene-2-carbonitrile Along with 5,6,7,8-tetrahydronaphthalene-1-carbonitrile isomer

A mixture of 5-bromo-1,2,3,4-tetrahydronaphthalene and 6-bromo-1,2,3,4 tetrahydronaphthalene (20 g), copper (I) cyanide (8.6 g) and anhydrous N-methylpyrrolidinone (41.3 g) were stirred under dry nitrogen at 130° C. for 40 h. The mixture was cooled to ambient temperature, further N-methylpyrrolidinone (10 g) was added along with saturated aqueous brine (30 ml), the resulting mixture was stirred at ambient for 3 hours and filtered to remove solids. The filtrates were extracted with n-hexane (3×50 ml). The combined organic extracts were washed with water (100 ml), dried (MgSO₄) and evaporated in vacuo to give crude product (16.2 g). This was purified by distillation to give 5,6,7,8-tetrahydronaphthalene-2-carbonitrile along with regioisomer (13.2 g, 95% purity, 84% yield).

### Preparation 12

### Conversion of 5-bromo-1,2,3,4-tetrahydronaphthalene and 6-bromo-1,2,3,4-tetrahydronaphthalene to 5,6,7,8-tetrahydronaphthalene-1-carboxylic acid and 5,6,7,8-tetrahydronaphthalene-rboxylic acid

n-Butyl lithium (9.6 ml of 2.5M solution in hexane) was added dropwise over 30 minutes to a stirred solution of 5-bromo-1,2,3,4-tetrahydronaphthalene in mixture with its regioisomer 6-bromo-1,2,3,4-tetrahydronaphthalene (5 g) in dry THF (125 ml) and hexane (35 ml) at −70° C., stirring was continued at −78° C. for 30 minutes, carbon dioxide gas was bubbled through the mixture at −70° C. until no further exotherm was evident, carbon dioxide gas addition was continued for a further 10 minutes as the reaction was allowed to warm to ambient temperature, the mixture was poured into 2M aqueous hydrochloric acid (100 ml) and the resulting mixture was extracted with diethyl ether (3×50 ml). The combined organic extracts were washed with water (100 ml) and were then extracted with 10% aqueous sodium carbonate solution (3×50 ml). The combined aqueous carbonate extracts were acidified carefully by addition of 2M hydrochloric acid to adjust the pH to pH 1. The resulting mixture was extracted with diethyl ether (3×50 ml), the combined organic extracts were washed with water (50 ml) and dried (MgSO₄) before solvent was removed by evaporation in vacuo to give the crude product in 64% yield comprising a mixture of regioisomers of 5,6,7,8-tetrahydronaphthalene carboxylic acid. This mixture was purified by repeated recrystallisation from ethyl acetate to give 5,6,7,8-tetrahydronaphthalene-2-carboxylic acid as crystallised solid in 93% purity along with 5,6,7,8-tetrahydronaphthalene-1-carboxylic acid as the major component present in the crystallisation mother liquors.

### Conversion of 5,6,7,8-tetrahydronaphthalene-2-carboxylic acid to 5,6,7,8-tetrahydronaphthalene-2-carbonitrile

Acetyl chloride (5 g, 64 mmol) is added dropwise to dry methanol (150 ml) with stirring at ambient temperature under dry nitrogen. Stirring is continued for 15 minutes, 5,6,7,8-Tetrahydronaphthalene-2-carboxylic acid (1 g, 5.7 mmol) is added, the mixture is stirred at ambient temperature for 10 hours and solvent removed by evaporation in vacuo to give methyl 5,6,7,8-tetrahydronaphthalene-1-carboxylate. This is then converted to 5,6,7,8-tetrahydronaphthalene-2-carbonitrile using the same procedure described above for conversion of methyl 4-bromo-2-naphthoate to 4-bromo-2-naphthonitrile using dimethylaluminium amide.

### Preparation 13

### Conversion of 4-bromo-5,6,7,8-tetrahydronaphthalene-2-carbonitrile to 4-bromo-2-naphthonitrile

4-Bromo-5,6,7,8-tetrahydronaphthalene-2-carbonitrile (0.1 g) was heated with 10% palladium on carbon (1.65 g) under air at 200° C. to 210° C. for 22 hours to give crude 4-bromo-2-naphthonitrile as seen by gc (approximately 75% yield by gc area).

### EXAMPLES

### Example 1

### Conversion of 4-bromo-2-naphthonitrile to 3-cyano-1-naphthoic acid via metallo-dehalogenation and carboxylation

To a 50 ml 4-neck round bottomed flask equipped with a magnetic stirrer, thermometer, septum, CO₂ inlet, N₂ inlet/bubbler and external dry ice/acetone cooling bath is charged (0.35 g, 1.25 mmol), anhydrous hexane (2 ml) and anhydrous THF (8 ml). The suspension is cooled to −75° C. and BuLi (0.6 ml) added dropwise over 20 min to the vigorously stirred suspension. The bright red solution is stirred for a further 5 min and then carbon dioxide bubbled very slowly through the reaction mixture with external cooling. The quenching reaction is very exothermic—maximum temperature reached is −65° C. Reaction is judged complete when no further temperature increase is observed upon addition of carbon dioxide. The mixture is stirred at −65° C. for a further 10 min, and then added cautiously to 2 M HCl. The product is extracted with ethyl acetate (3×50 ml), the combined extracts dried (MgSO₄) and the solvent removed in vacuo to give 3-cyano-1-naphthoic acid (circa 20% yield).

¹H nmr (D₆DMSO): 7.69-7.87 (m, 2H, 2×ArH), 8.14 (d, 1H, J=7.9 Hz, ArH), 8.28 (d, 1H, J=1.5 Hz, ArH), 8.79 (s, 1H, ArH), 8.85 (d, 1H, J=8.4 Hz, ArH).

MS: 197 (M⁺), 180, 152, 125, 29, 18.

### Example 2

### 3-cyano-1-naphthoic acid via carbonylation

Bis(triphenylphosphine)palladium (II) chloride (0.77 g) in N-methylpyrrolidinone (170 g), (10 g), triphenyl phosphine (0.57 g), and triethylamine (11 g,) are mixed in a nitrogen inerted pressure vessel (Parr reactor) at ambient temperature. Water (15.5 g) is added and the reactor is repeatedly purged with argon to remove residual air or oxygen. The reactor is vented and then pressurised with carbon monoxide to 7 bar absolute pressure (6 bar gauge pressure) and the mixture stirred at 85 C for 10 hours, maintaining carbon monoxide pressure within the reactor at 6 barg. The mixture is cooled to 50 C and vented to atmospheric pressure, and the reaction mixture then filtered through a bed of celite to remove solids. The filter cake is washed with toluene (160.5 g) and then with water (124 g). The combined filtrates and washes are allowed to settle and the lower aqueous layer separated. The toluene layer is extracted with water (2×124 g). The combined aqueous phase and aqueous extracts were washed with toluene (120 g), 2M hydrochloric acid (64.5 ml) are added to the aqueous solution over 30 minutes with stirring at 25 to 30 C. The organic layer is separated off and retained, the aqueous layer is extracted with toluene (2×120 g). The combined organic layer and toluene extracts are mixed with water (62 g) and 2M sodium hydroxide solution (16.2 ml) to extract the product into the aqueous phase. The organic phase is extracted with further water (62 g) plus 2M sodium hydroxide solution (16.2 ml). The combined aqueous extracts are mixed with dichloromethane (350 g) and the mixture acidified by addition of 2M hydrochloric acid (43 ml) over 30 minutes at 25 to 30 C. The lower organic phase is separated and retained, the aqueous phase is extracted with further dichloromethane (100 g). The combined dichloromethane solution and extract are washed with 2M hydrochloric acid (21.5 ml), toluene (120 g) is added and dichloromethane is removed by evaporation under reduced pressure to leave a toluene solution of the product. This solution is heated to 60 C, iso-hexane (300 g) is added over 30 minutes at 60 C, and the mixture cooled over 3 hours to 5 C so as to crystallise the product, which is isolated by filtration. The product is washed with pre-cooled iso-hexane at 0 C to 5 C and it is then dried overnight in a vacuum oven at 40 C (5.66 g, 65% yield).

The obtained product is confirmed by analysis to be the same as in Example 1.

### CONCLUSIONS

The new routes described herein offer significantly improved means for large scale manufacture of naphthalene cyanoacid (1) compared with methodology available from the chemical literature. These new routes offer advantage in terms of significantly improved through-route yield (with considerable potential for yet further yield improvement), they avoid the large scale process operability difficulties associated with the previous literature chemistry, they give product of lower cost of manufacture and they avoid the effluent toxicity and reagent toxicity associated with use of stoichiometric mercury salts specified in the previously published chemistry to such products.

1. A process for preparing a compound of formula (1) 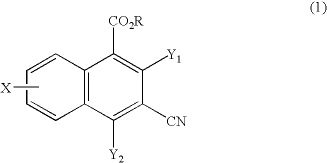
wherein X, and/or Y₁, and Y₂ are independently H, cyano, nitro, trifluoromethoxy, trifluoromethyl, alkoxy, or alkyl, and R is H or alkyl, wherein
a) when R═H, the process comprises metallo-dehalogenation followed by carboxylation of a compound of formula (12) 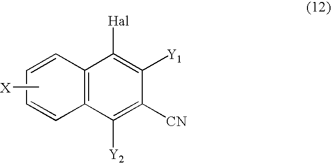
wherein X, Y₁ and Y₂ are as defined above, and Hal is Br, I or Cl; or
b) when R═H or alkyl, the process comprises palladium-mediated carbonylation of a compound of formula (12) 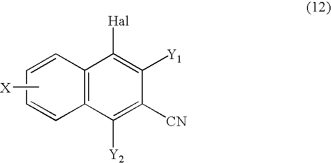
with the proviso that the compound 1-iodo-3-cyano-2-methoxynaphthalene is excluded, followed by solvolysis of the carbonylated compound.

2. The process for preparing the compound of formula (1) according to claim 1 wherein, step a) is carried out by treatment of the compound of formula (12) with an alkyl-lithium reagent followed by reaction of the lithiated intermediate with carbon dioxide and then acidification.

3. A compound of formula (12), 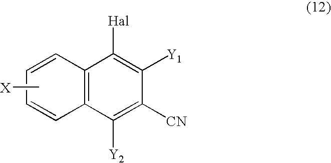
wherein X, Y₁ and Y₂ are independently H, cyano, nitro, trifluoromethoxy, trifluoromethyl, alkoxy, or alkyl and Hal is Br, I or Cl, with the proviso that the compounds 1-iodo-3-cyano-2-methoxynaphthalene and 1-chloro-3-cyano-2-methoxynaphthalene are excluded.

4. The process according to claim 1, for preparing a compound of formula (12, Y₁═Y₂═X═H), 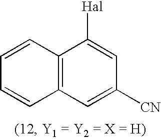
wherein Hal is Br, I or Cl,
wherein the process comprises a reaction sequence selected from the group consisting of:
(i)
(a) treating malic acid with strongly acid dehydrating media to give coumalic acid;
(b) esterifying coumalic acid to give a pyrone ester;
(c) brominating the pyrone ester to give a 3-bromo coumalic ester;
(d) reacting the 3-bromo coumalic ester (10) with in situ generated benzyne followed by decarboxylation to give a bromonaphthoate (11); and
(e) converting the bromonaphthoate (11) to 1-bromo-3-cyano naphthalene;
(ii)
(a) treating malic acid with a strongly acid dehydrating media to give coumalic acid;
(b) converting coumalic acid into coumalonitrile and subsequently brominating to give 3-bromo-5-coumalonitrile; and then
(c) converting 3-bromo-5-coumalonitrile into 1-bromo-3-cyano naphthalene
by cycloaddition of in situ generated benzyne, followed by subsequent decarboxylation and (iii)
(a) cyanation of 1,2,3,4-tetrahydronaphthalene followed by bromination to give compound (63) 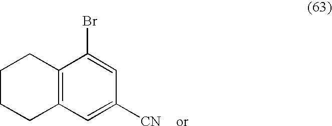
(b) bromination of 1,2,3,4-tetrahydronaphthalene followed by cyanodebromination, followed by bromination to give the compound of formula (63); or
(c) bromination of 1,2,3,4-tetrahydronaphthalene followed by carboxylation followed by conversion to the 6-cyano-1,2,3,4-tetrahydronaphthalene followed by bromination to give compound (63); followed by
c1) oxidative aromatization of compound (63) into 1-bromo-3-cyano naphthalene.

5. The process according to claim 4, wherein the reaction of the ester with benzyne in step (d) of process (i) generates an intermediate having structure (15) 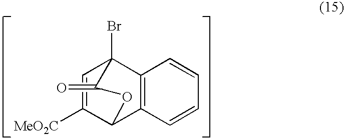
which intermediate is decarboxylated to give the bromonaphthoate.

6. The process according to claim 4, wherein step (e) of process (i) is carried out by means selected from the group consisting of:
e1) reaction of the bromonaphthoate compound with ammonia to give compound (18) 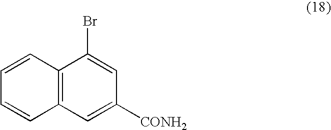
followed by dehydration to give the cyano compound;
e2) reaction of the bromonaphthoate compound with hydroxylamine or a salt thereof to give compound (20); 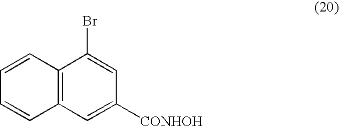
followed by dehydration to give the cyano compound; and
e3) adding a solution of Me₂AlNH₂ to a solution of the bromonaphthoate compound in a high-boiling solvent and heating the mixture to reflux, followed by rapid conversion to the cyano compound.

7. The compound of formula (20)

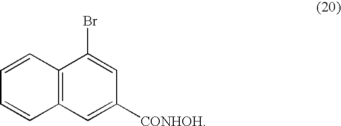

8. The compound of formula (18)

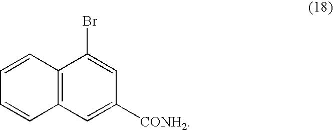

9. The compound which is 3-bromo-5-coumalonitrile.

10. The compound of formula (63)

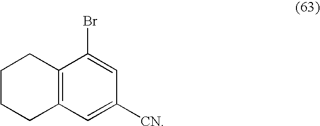

11. The process according to claim 4, wherein the strongly acid dehydrating media is oleum.

12. The process according to claim 6, wherein the high-boiling solvent is m-xylene.

13. The bromonaphthoate compound of formula (11), [chemical structure]
